(19) United States
(12) Patent Application Publication
(10) Pub. No.: US 2012/0078342 A1
(43) Pub. Date: Mar. 29, 2012

(54) POSITIONING CATHETERS USING IMPEDANCE MEASUREMENT

(75) Inventors: Michael Vollkron, Pressbaum (AT); Michael Lippert, Ansbach (DE)

(73) Assignee: BIOTRONIK SE & CO. KG, Berlin (DE)

(21) Appl. No.: 13/232,290

(22) Filed: Sep. 14, 2011

Related U.S. Application Data
(60) Provisional application No. 61/385,567, filed on Sep. 23, 2010.

(51) Int. Cl. A61F 2/84

(52) U.S. Cl. 623/1.11

(57) ABSTRACT

The invention relates to a method for determining the position of a catheter in a blood vessel relative to a change in the vascular cross section, comprising the steps: providing a catheter that includes at least two electrodes which can be brought into electrically conductive contact with the surrounding blood while the catheter is being positioned in the blood vessel, wherein the two electrodes are installed on the catheter along the longitudinal axis at a defined distance from each other; advancing the catheter in the vessel to be treated, toward the change in the vascular cross section; and measuring the impedance across the two electrodes while the catheter is being advanced; and to a catheter for use in a method of this type.

### CROSS REFERENCE TO RELATED APPLICATION

This patent application claims the benefit of U.S. Provisional Patent Application No. 61/385,567, filed on Sep. 23, 2010, which is hereby incorporated by reference in its entirety.

### TECHNICAL FIELD AND BACKGROUND

Angioplasty, or percutaneous transluminal angioplasty (PTA) or percutaneous transluminal coronary angioplasty (PTCA), is a method for expanding or reopening blood vessels that have become constricted or closed (usually arteries, and to a lesser extent veins). Common methods of angioplasty are balloon dilation and the application of stents.

In the fields of interventional radiology, cardiology, and angiology, balloon dilation is understood, within the scope of angioplasty, to mean a method for expanding pathologically constricted blood vessels using a balloon catheter i.e. a vascular catheter with a balloon installed thereon that is expanded slowly under high pressure (6-20 bar) once it has reached the constricted site. The constrictions, which result primarily due to atherosclerotic changes (angiosclerosis), are therefore expanded in a manner such that they no longer hinder blood flow, or do so to a lesser extent.

Balloon catheters are nearly always inserted into the groin and moved to the stenosis (constriction) using a guide wire and guide catheter, and are then expanded using pressure. This usually eliminates the constriction, thereby avoiding the need for surgery.

Stent implantation has become established as one of the most effective therapeutic measures for treating vascular disease. Stents are used to provide support in a patient's hollow organs. To this end, stents of a conventional design have a tubular base body with a filigree support structure composed of metallic struts; the tubular base body is initially present in a compressed form for insertion into the body, and is expanded at the application site. One of the main applications of stents of this type is to permanently or temporarily widen and hold open vasoconstrictions, in particular constrictions (stenoses) of the coronary arteries. In addition, aneurysm stents are known, for example, which are used to support damaged vascular walls.

Stents include a circumferential wall having a support force that suffices to hold the constricted vessel open to the desired extent; stents also include a tubular base body through which blood continues to flow without restriction. The circumferential wall is typically formed by a latticed support structure that enables the stent to be inserted, in a compressed state having a small outer diameter, until it reaches the constriction in the particular vessel to be treated, and to be expanded there, e.g. using a balloon catheter, to the extent that the vessel finally has the desired, increased inner diameter. The cardiologist must monitor the process of positioning and expanding the stents during the procedure, and the ultimate position of the stent in the tissue once the procedure has been completed.

In regards to balloon dilation and the application of stents, therapeutic success depends substantially on the correct positioning of the balloon or the stent relative to the constriction of the blood vessel to be treated. It is therefore extremely important to determine the position of the catheter as quickly and exactly as possible while the catheter is being brought into position and before the vessel is expanded for therapeutic purposes i.e. the actual expansion of the stent or balloon, to ensure that the therapeutic expansion of the vessel is performed at the desired point in the vessel. Although catheters and methods that spare the patient additional exposure to radiation have already been proposed for use to control and expand stents or balloons, see e.g. US 2010/0010612 A1, the stent is positioned before expansion mainly using known imaging methods such as radiography. The previous imaging methods have a few disadvantages, such as a lag period in the method, insufficient accuracy, additional costs, stressing the patient e.g. via the dosage of radiation, to name a few.

### SUMMARY

The feature of the present invention is to lessen or prevent one or more disadvantages of the prior art. In particular, new ways to position a catheter in a blood vessel shall be provided.

This feature is realized by providing a method for determining the position of a catheter in a blood vessel relative to a change in the vascular cross section, in particular a constriction or expansion of the blood vessel, comprising the steps:
providing a catheter that includes at least two electrodes which can be brought into electrically conductive contact with the surrounding blood while the catheter is being positioned in the blood vessel, wherein the two electrodes are installed on the catheter along the longitudinal axis at a defined distance from each other;
advancing the catheter in the vessel to be treated, toward the change in the vascular cross section; and
measuring the impedance across the two electrodes while the catheter is being advanced.

The method according to the invention is based on the surprising finding that the dependence of impedance, which is measured across two electrodes of a catheter, on the vascular cross section can be utilized to determine the position of the catheter in a blood vessel relative to a change in the vascular cross section, in particular relative to a constriction or expansion of the blood vessel. While the catheter is being advanced in the blood vessel, a correlation results between the impedance that is measured across the two electrodes, and the vascular cross section of the vascular region that has been reached or is being passed through by the region of the catheter on which the electrodes are disposed. If the vascular cross section decreases, then an increase in the impedance can be measured; if the vascular cross section increases, a decrease in the impedance can be measured. If the impedance that is measured is plotted against the advance of the catheter, the changes in impedance may be used directly to determine the position of the catheter relative to a change in the vascular cross section. The graph of measured impedance against the advance of the catheter can be used to determine the position of the catheter and to infer the position of devices connected to the catheter, such as stents mounted or crimped thereon, or dilatable balloon regions. The method according to the invention is therefore also suitable, in particular, for positioning stents or dilatable balloons before expansion. It is also possible to determine the extent of a change in the vascular cross section, in particular a constriction or an expansion, in a blood vessel along the longitudinal axis of the blood vessel.

The method according to the invention makes it possible to determine the position of a catheter in a blood vessel relative to a change in the vascular cross section, in particular a constriction or expansion of the blood vessel. Therefore, the absolute spatial position of the catheter is not determined directly, but rather its location in the blood vessel relative to the change in the vascular cross section in this blood vessel. The method according to the invention can be combined with other methods and thereby also help to determine the absolute spatial position of a catheter. Moreover, various derived quantities can be detected using impedance measurement, such as: heart rate, changes in the vascular state along the direction of the advance of the catheter, the respiration rate when the catheter is located in the pulmonary circulation, or pulse transit times (using at least three electrodes).

In this context, a change in the vascular cross section refers to any constriction and/or expansion of the cross section of a blood vessel, independently of whether it is a pathological change in the vessel or not. In particular, the change in the vascular cross section can be one or more radially symmetrical and/or eccentric stenoses/aneurysms.

The method according to the invention can be used likewise to position the catheter relative to a previously installed, electrically conductive stent (e.g. to “redilate” a stent that was not sufficiently dilated). Other graphs of impedance result that likewise provide unambiguous inferences regarding the position of the catheter relative to the installed stent.

First, a catheter having at least two electrodes is provided. Any catheter type or technology can be used, provided that the catheter includes two electrodes and is designed such that impedance can be measured and determined across the two electrodes. The two electrodes are installed on the catheter in a manner such that they have electrically conductive contact with the surrounding blood while the catheter is being positioned in the blood vessel. The catheter and the electrodes are designed such that a defined voltage can be applied to the electrodes, e.g. using specially designed guide means of the catheter, and the variation in conductivity across the electrodes can be measured. Suitable catheters and electrode assemblies are described, as examples, in US 2010/0010612 A1, for instance. In particular, the electrodes of the catheter can be designed such that they can be connected in an electrically conductive manner to a voltage source and an ampere meter, or to a current source and a voltmeter. The electrodes can be contacted e.g. via conductive coatings to the inside of the catheter lumen. If the catheter lumen should be filled with an electrically conductive fluid while it is being advanced, it can also be used to contact the electrodes. As an alternative or in addition thereto, the electrodes can also be electrically contactable via the guide wire of the catheter. The electrical contacting of the two electrodes can also be established using lines or coatings that are formed or integrated specially in the catheter wall.

The two electrodes are disposed on the catheter at a defined distance from each other along the longitudinal axis of the cathether, preferably close to the end of the catheter that is inserted into the blood vessel. The distance between the two electrodes can be selected such that a stent can be installed or crimped between the two electrodes, and/or the catheter has a dilatable region e.g. a deflatable balloon. If the catheter should carry a stent between the two electrodes, the stent can be fastened to the catheter in a manner such that the stent is not connected to any electrode of the catheter in an electrically conductive manner, although the stent itself can be electrically conductive. While the catheter is being advanced in the blood vessel, the stent is preferably in contact with the surrounding blood vessel in a manner such that electrically conductive contact between the stent and the surrounding blood is made possible.

The catheter can also have more than two electrodes for measuring impedance. If e.g. a plurality of electrodes is used on different lateral surfaces of the catheter, e.g. four electrodes, with two electrodes each on opposite sides of the catheter, it is possible to reduce the influence of an undesired contact with the vessel wall on the impedance signal that is measured.

In the method according to the invention, once the catheter has been made available, it is inserted into the vascular system and, in the blood vessel to be treated, is advanced toward the change in the vascular cross section, in particular toward the constriction or expansion of the vessel. This advance can be carried out using the usual guide means of the catheter. There are basically no special requirements on the guide means of the catheter. Suitable guide means are known to a person skilled in the art. It is advantageous, however, for the catheter to include means or to be combinable with means that enable the catheter to be advanced as accurately as possible. Preferably, means can be used for this purpose that measure the extent of advancement as exactly as possible. In particular, these means can be designed such that the advance can be regulated and/or controlled directly using these means, or may be carried out in an automated manner.

In the method according to the invention, the impedance across the two electrodes is measured while the catheter is being advanced. Impedance can be measured continuously or discontinuously at predefined intervals. To this end, a measuring current is applied across the two electrodes, and the voltage drop between the two electrodes is measured, thereby enabling the impedance to be determined. Devices and methods for measuring impedance, or preferably to detect and depict impedance as a function of advancement, are known to a person skilled in the art and are merely structural adaptations and the suitable configuration of existing devices that a person skilled in the art can design within the scope of routine activities. To measure impedance, it is preferable to apply the lowest measuring currents possible; particularly preferably, measuring currents in the range of 1 μA to 1 mA are used, in particular in the form of pulses having pulse widths in the range 10 μs to 1000 μs, or as alternating current having a frequency in the range of 1 kHz to 100 kHz. The combination should be selected such that an effective stimulation of tissue, in particular the cardiac muscle and nerve fibers, by the measurement pulses is ruled out.

In the method according to the invention, a measuring device can be used to measure impedance. The measuring device used to measure impedance is connected or connectable to the catheter in a manner such that the impedance across the two electrodes of the catheter can be measured. The measuring device can be operated e.g. using batteries, rechargeable batteries, or a suitable power supply unit. The measuring device is preferably designed such that it can be reused for several applications, although it can also be designed as a disposable device. The measurement result can be output visually, e.g. as a diagram, and/or acoustically e.g. as a sound or a varied pitch. The impedance meter can also be provided with, or combined with, further measuring devices e.g. the measuring device can be equipped with additional devices for determining the position of the catheter, in particular for showing an image thereof. The impedance meter can be designed and configured such that the measurement data used to determine the position of the catheter are evaluated automatically. This evaluation can be presented and/or communicated to the physician in a manner such that the physician is notified acoustically and/or visually regarding suggestions for the optimal positioning of the catheter, and/or is alerted to the fact that the stent is too short or too long.

If the impedance meter is combined e.g. with a mechanical, controllable advancing device, preferably an advancing device having force feedback, then fully automated fine-positioning of the catheter can be achieved.

The method according to the invention delivers a graph of impedance during advancement, the graph being easy to interpret. Preferably, the variation of impedance can be plotted as impedance versus the extent of advancement, and can be used to determine position. The variation of impedance that is measured during advancement is used to determine the position of the catheter in the blood vessel along the longitudinal axis of the blood vessel and relative to a change in the vascular cross section. An increase in impedance indicates that one of the two electrodes has reached a region having a smaller vascular cross section, while a decrease in impedance implies that one of the electrodes has reached a region having a larger vascular cross section, or has left a region having a smaller vascular cross section. Since the variations in impedance that are measured are composed of an addition of the states at each of the two electrodes, a graph of impedance can be used to directly determine whether neither of the electrodes, or one or two of the electrodes is disposed in the region of a vascular constriction or expansion, that is, a region in which the vascular cross section has changed. Since the distance between the two electrodes on the catheter is known, the method according to the invention can be used to determine the extent of a blood vessel constriction or expansion along the longitudinal axis of the blood vessel. Since the method according to the invention can be used, in general, to identify and characterize regions in a blood vessel having a cross section that has changed, the method can also be used to monitor the therapeutic success of a stent application or balloon dilation.

The method according to the invention can also be used during placement of a stent in a blood vessel, in particular to determine the optimal position before the stent is expanded.

The catheter to be used in the method according to the invention can carry a stent between the two electrodes. The catheter can then be positioned in the blood vessel, using the method according to the invention, such that the stent comes to rest in the region of the constriction and is then applied there via expansion.

According to a preferred embodiment, a catheter carries a conductive stent between the two electrodes. The electrically conductive stent is contacted in an electrically conductive manner with the surrounding blood while the catheter is being positioned in the blood vessel. As a result, the entry and exit of each of the two electrodes into or out of the region of the change in vascular cross section results, in each case, in a stepwise change in impedance across the two electrodes. In particular, the entrance of each of the two electrodes into the region of a constriction of the blood vessel independently of each other results in a stepwise increase in impedance, and the exiting of each of the two electrodes out of the region of constriction of the blood vessel results in a stepwise decrease in impedance across the two electrodes. This is due to the fact that the current only has to travel a short path from the electrodes through the surrounding tissue to the stent, where it resumes flowing. Therefore, when a conductive stent is used, the impedance that is measured is based soley on the electrical properties of the tissue surrounding the two transition regions between the electrodes and the stent (vascular wall/blood). On the basis of the variation in impedance it can therefore be unequivocally stated whether no electrodes, or one or both electrodes are located in a region having a narrower vascular cross section at a certain point during the advancement, that is, when the catheter has reached a certain point along the longitudinal axis of the blood vessel. On the basis of the graph of impedance versus advancement it can therefore be determined directly whether the stent, which is located between the two electrodes, is located in the region of the constriction to be treated, or not. This knowledge can be used in the method according to the invention. It is therefore possible to position the catheter in the blood vessel relative to a change in the vascular cross section such that the impedance across the two electrodes is located between the stepwise impedance change of the first electrode, which has already emerged from the region of the change in vascular cross section, and in front of the stepwise impedance change caused by the second electrode which is entering the region of the change in vascular cross section. In particular, it is possible to position the catheter in the blood vessel relative to a constriction such that the impedance across the two electrodes is located between a stepwise decrease in impedance of the first electrode, which has already emerged from the region of the constriction, and in front of a stepwise increase in impedance caused by the second electrode which is entering the region of the constriction. If this is the case, then the stent is located in the region of a constriction of the vascular cross section. If the catheter is placed substantially in the middle between the stepwise decrease in impedance of the first electrode and the stepwise increase in impedance of the second electrode, then the stent is located in the center of the constriction and can be placed there via expansion.

The method described herein can be used analogously to locate a local expansion of the vessel. The variations in impedance are reversed in that case.

In another embodiment of the method according to the invention, a catheter is provided that does not carry a stent, or carries a substantially non-conductive stent, or the stent is not in electrically conductive contact with the surrounding blood while the catheter is being positioned in the blood vessel. As a result, the entry and exit of each of the two electrodes into or out of the region of the change in vascular cross section results, in either case, in a ramp-type change in impedance across the two electrodes. In particular, the entrance of each of the two electrodes into the region of a constriction of the blood vessel can result in a ramp-shaped increase in impedance across the two electrodes, and the emergence of each of the two electrodes from the region of constriction of the blood vessel can result in a ramp-shaped decrease in impedance across the two electrodes. This is due to the fact that, in this case, the surrounding tissue also contributes to the impedance that is measured along the entire length between the two electrodes, even though the direct proximity of the electrodes is weighted more heavily. The impedance graphs that have a “ramp-shaped” schematic appearance therefore need not proceed linearly, although they always show a continuously increasing or decreasing graph, and not just a step. On the basis of the variation in impedance it can therefore be unequivocally stated once more whether no electrodes, or one or both electrodes are located in a region having a narrower vascular cross section at a certain point during the advancement i.e. when the catheter has reached a certain point relative to the longitudinal axis of the blood vessel. On the basis of the graph of impedance versus advancement it can therefore be deter pined directly whether the stent, which is located between the two electrodes, is located in the region of a constriction to be treated, or not. This knowledge can be used in the method according to the invention. It is therefore possible to position the catheter in the blood vessel in a manner such that the variation in impedance is situated at an impedance plateau located between a ramp-shaped increase in impedance and a ramp-shaped decrease in impedance. If this is the case, then the stent is located in the region of the constriction. If the catheter is placed substantially in the center of the plateau, in the graph of impedance, then the stent is located in the center of the constriction, and can be placed there in a particularly advantageous manner, via expansion.

This method can also be used analogously to locate a local expansion of the vessel. The variations in impedance are reversed in this case.

The method according to the invention can therefore also be used to determine whether a stent also has a sufficient length to effectively expand or stabilize the constriction or expansion to be treated along the entire extension in the longitudinal axial direction.

The method according to the invention does not require the use of x-rays, thereby ensuring that, overall, the patient is exposed to a lower radiation dose or none at all when a stent is applied. The method according to the invention tracks the advance of the catheter in real time and, overall, does not lengthen the treatment time.

Given that the method according to the invention makes it possible to locate the optimal position for the application of the stent or the balloon before expansion is performed, the reliability of the vascular expansion and stabilization by stents or balloon catheters is improved overall.

Since positioning can be performed in an optimal manner, combined with the fact that an incorrect stent length can be detected at an early point in time, the use of a method according to the invention results in improved therapeutic success overall.

The method according to the invention can also be used to identify vascular branchings e.g. to identify suitable implantation sites e.g. for the implantation of pressure sensors, pacemaker electrodes, defibrillator electrodes, or CRT electrodes.

The present invention also relates to a catheter for use in a method according to the invention.

### DESCRIPTION OF THE DRAWINGS

FIG. 1 shows a schematic depiction of a catheter for use in a method according to the to invention.

FIG. 2 shows a comparison of impedance graphs for different distances between the two electrodes, measured for a catheter having a conductive stent. (A) graph of impedance versus advancement when the distance between the electrodes of the catheter is smaller than the range of the constriction; (B) graph of impedance versus advancement when the distance between the electrodes of the catheter is identical to the range of the constriction; (C) graph of impedance versus advancement when the distance between the electrodes of the catheter is greater than the range of the constriction.

FIG. 3 shows a comparison of impedance graphs for different distances between the two electrodes, measured for a catheter having a non-conductive stent, or only a balloon. (A) graph of impedance versus advancement when the distance between the electrodes of the catheter is smaller than the range of the constriction; (B) graph of impedance versus advancement when the distance between the electrodes of the catheter is identical to the range of the constriction; (C) graph of impedance versus advancement when the distance between the electrodes of the catheter is greater than the range of the constriction. The ramp-shaped regions, which are drawn here schematically as straight lines, can also assume a shape in reality that is non-linear but is continuously increasing or decreasing nonetheless.

The invention is explained in greater detail below with reference to embodiments.

### DETAILED DESCRIPTION

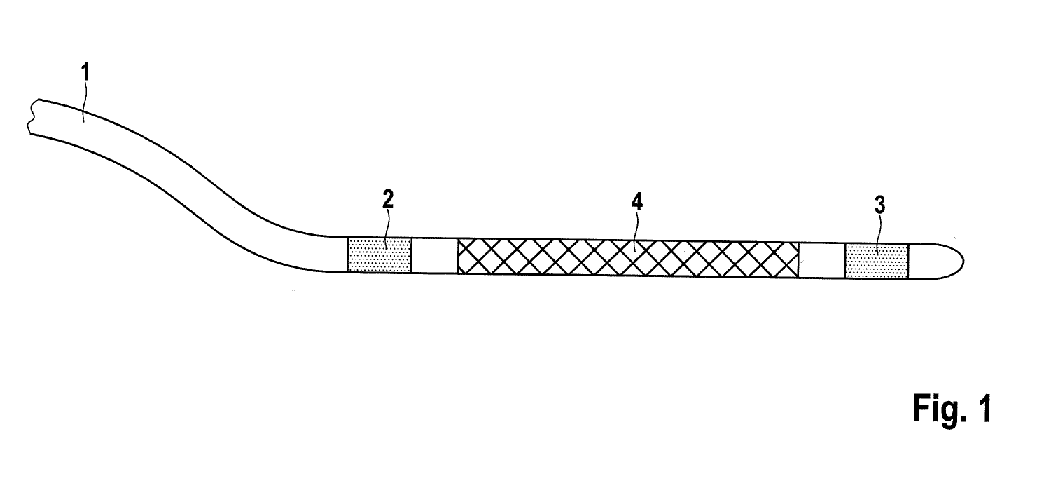

A catheter that can be used in a method according to the invention is depicted schematically in FIG. 1. The catheter includes a base body 1 and has two electrodes, 2 and 3, close to the end that is introduced into the blood vessel. Electrodes 2 and 3 are disposed on base body 1, separated by a distance in the longitudinal axial direction. A stent 4 is placed between electrodes 2 and 3; once stent 4 has been positioned, it can be applied in the blood vessel via expansion. This stent can be in electrically conductive contact, or not, with the surrounding blood while the catheter is being positioned in the blood vessel.

The catheter shown in FIG. 1 can be positioned using a method according to the invention such that stent 4 is located in an optimal position in the region of a stenosis. For this purpose, the catheter is first introduced into the vascular system, and is then advanced in a suitable manner toward the constriction in the blood vessel to be treated. During the advancement, a measuring current is applied, and the impedance across electrodes 2 and 3 is measured continuously. On the basis of the graph of impedance versus advancement it is possible to determine the position of the catheter relative to a constriction (stenosis) in the blood vessel. As soon as the catheter is positioned accordingly, stent 4 can be applied via expansion, thereby expanding the constriction.

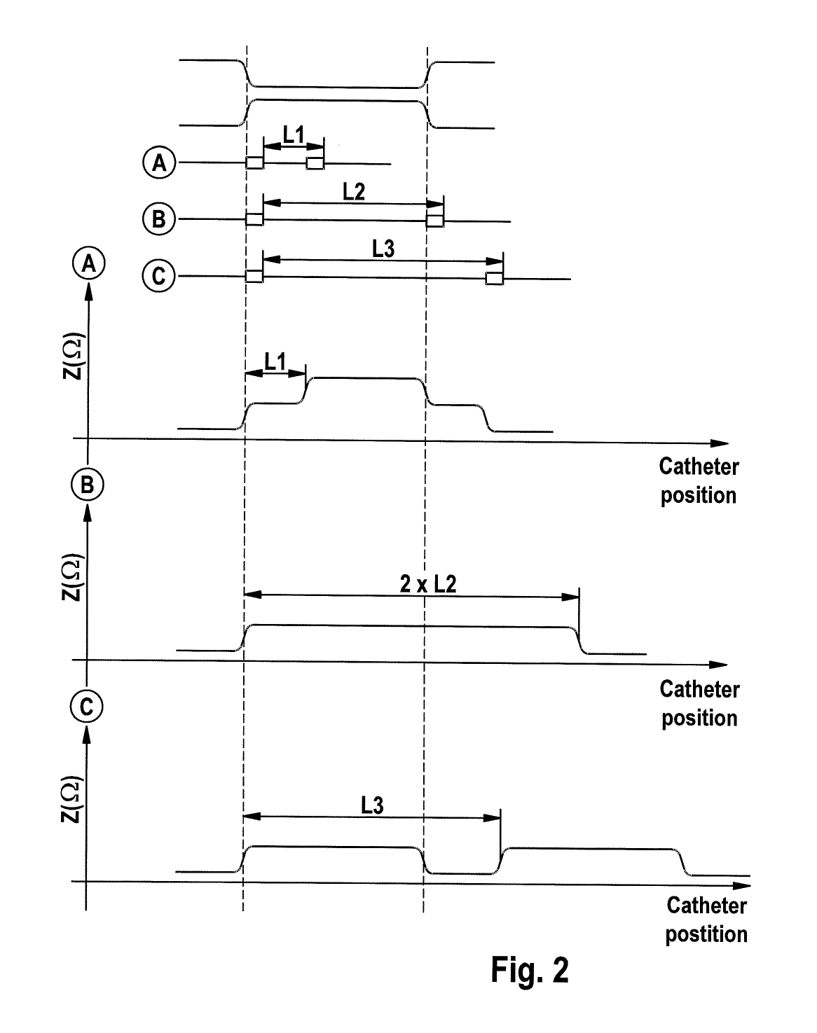

If stent 4 is conductive and is in direct contact with blood, the measuring current flows mainly across the stent body, and the main changes in impedance to be measured occur at the two transition regions between electrodes 2, 3 and stent 4. It is therefore possible to determine the position of the catheter relative to the constriction, and therefore the position of stent 4, on the basis of the graph of impedance shown in FIG. 2. If the distance between electrodes 2 and 3 is shorter than the longitudinal axial extension of the constriction to be treated, then the impedance graph has two steps (A). A first stepwise increase in impedance indicates that electrode 3 has entered the region of the constriction. A second stepwise increase in impedance, which is added thereto, indicates that electrode 2 has entered the region of the constriction. The impedance plateau between the first and the second stepwise increase in impedance corresponds to the distance between electrodes 2 and 3. Upon emergence from the region of the constriction, the complementary image for the stepwise decreases in impedance results. For the case in which the distance between the electrodes corresponds to the length of the constriction, a single stepwise increase in impedance results, followed by a plateau in impedance, the length of which corresponds to twice the distance between electrodes 2 and 3. However, if the distance between the electrodes is greater than the length of the constriction, then a first stepwise increase in impedance that occurs when electrode 3 enters the region of the constriction is followed by an impedance plateau having the length of the constriction. This impedance plateau is followed by a stepwise decrease in impedance, which indicates that electrode 3 has emerged from the constricted region. Impedance increases once more when electrode 2 enters the region of the constriction. The impedance plateau between the stepwise decrease in impedance of electrode 3 and the stepwise increase in impedance of electrode 2 indicates the length by which the distance between electrodes 2 and 3 exceeds the longitudinal axial extension of the constriction. If the catheter is positioned such that the impedance is located between the first decrease in impedance and the second increase in impedance, then it can be assumed that stent 4 is located in the region of the constriction and extends along the entire region to be treated. The catheter is therefore located in a position in which stent 4 can be applied via expansion. The ideal position for the application of stent 4 is therefore in the middle between the first decrease in impedance and the second increase.

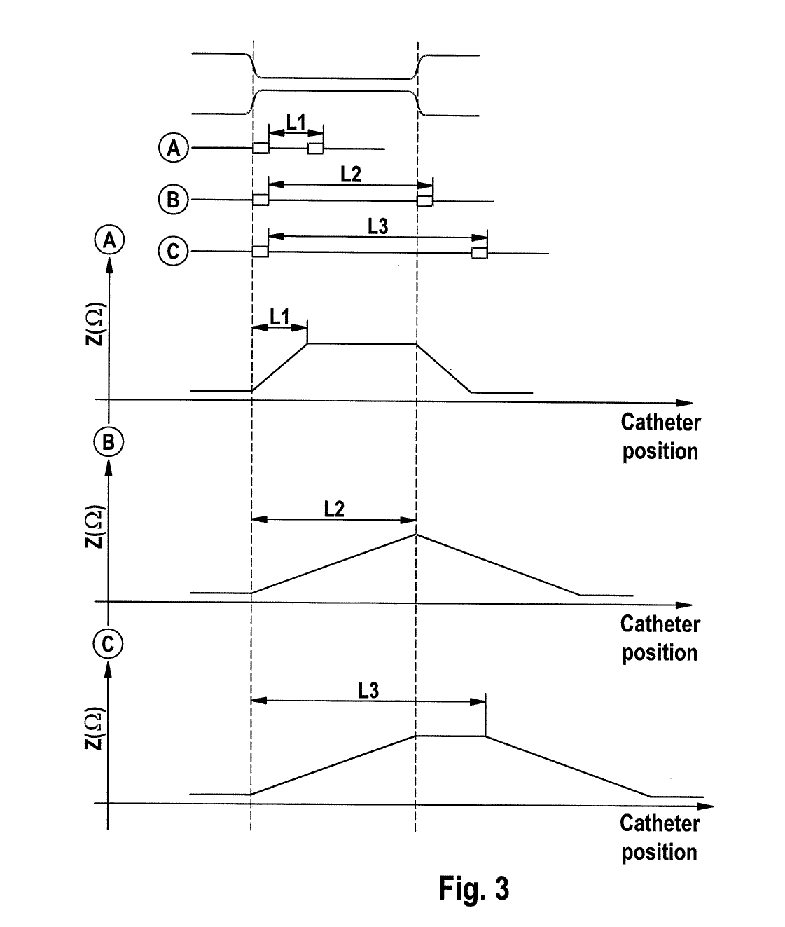

If stent 4 is not conductive, or if stent 4 is not in electrically conductive contact with blood while the catheter is being advanced, then the patterns of the impedance graphs change, but the catheter can still be positioned unequivocally and the length of the constriction can be checked, as depicted in FIG. 3. If the distance between electrodes 2 and 3 is shorter than the constriction to be treated, a ramp-shaped increase in impedance (A) results, which starts when electrode 3 enters the constriction, and ends when electrode 2 has likewise fully entered the region of the constriction. The length of this ramp corresponds to the distance between electrodes 2 and 3. If the distance between electrodes 2 and 3 is identical to the length of the constriction, then the graph of impedance assumes the shape of a triangle (B). If the distance between electrodes 2 and 3 is longer than the constriction, then a ramp forms that has the same length as the constriction to be treated (C). This is followed by an impedance plateau, the length of which is identical to the difference between the length of the constriction and the distance between electrodes 2 and 3. If the catheter is positioned such that the impedance in this impedance plateau is located between the ramp-shaped increase in impedance and the subsequent ramp-shaped decrease in impedance, then it can be assumed that stent 4 is located in the region of the constriction and extends along the entire region to be treated. The catheter is therefore located in a position in which stent 4 can be applied via expansion. The ideal position for the application of stent 4 is therefore in the middle of this impedance plateau.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

What is claimed is:

1. A method for determining a position of a catheter in a blood vessel relative to a change in a vascular cross section, in particular a constriction or expansion of the blood vessel, comprising the steps:
a) providing a catheter that includes at least two electrodes which can be brought into electrically conductive contact with surrounding blood while the catheter is being positioned in the blood vessel, wherein the two electrodes are installed on the catheter along a longitudinal axis at a defined distance from each other;
b) advancing the catheter in the vessel to be treated, toward the change in the cross section; and
c) measuring the impedance across the two electrodes while the catheter is being advanced.

2. The method according to claim 1, wherein the variation in impedance measured during advancement is used to determine the position of the catheter in the blood vessel along the longitudinal axis of the blood vessel and relative to the change in the vascular cross section.

3. The method according to claim 1, wherein the change in impedance across the two electrodes is determined continuously or discontinuously.

4. The method according to claim 1, wherein a graph of impedance is determined as impedance as a function of an extent of advancement.

5. The method according to claim 1, wherein the catheter includes a dilatable region between the two electrodes.

6. The method according to claim 1, wherein the catheter includes a stent between the two electrodes.

7. The method according to claim 6, wherein the catheter is positioned in the blood vessel such that the stent can be applied in a region of the change in the vascular cross section.

8. The method according to claim 6, wherein the catheter carries a conductive stent which can be contacted in an electrically conductive manner with the surrounding blood while the catheter is being positioned in the blood vessel.

9. The method according to claim 8, wherein the entry and exit of each of the two electrodes into or out of the region of the change in vascular cross section results, in each case, in a stepwise change in impedance across the two electrodes.

10. The method according to claim 9, wherein the catheter is positioned in the blood vessel such that the impedance across the two electrodes is located between the stepwise impedance change of the first electrode, which has already emerged from the region of the change in vascular cross section, and in front of the stepwise impedance change caused by the second electrode which is entering the region of the change in vascular cross section.

11. The method according to claim 5, wherein the catheter does not carry a stent, carries a stent that is substantially non-conductive, or the stent cannot be contacted in an electrically conductive manner with the surrounding blood while the catheter is being positioned in the blood vessel.

12. The method according to claim 11, wherein the entry and exit of each of the two electrodes into or out of the region of the change in vascular cross section results, in each case, in a ramp-shaped change in impedance across the two electrodes.

13. The method according to claim 12, wherein the catheter is positioned in the blood vessel such that the course of impedance is situated in an impedance plateau located between a ramp-shaped increase in impedance and a ramp-shaped decrease in impedance.

14. The use of a method according to claim 1 to place a stent in a blood vessel, to determine the extent of a change in the vascular cross section, and/or to monitor the therapeutic success after a constriction or expansion of the blood vessel has been expanded and/or stabilized via balloon dilation or stent application.

15. A catheter for use in a method according to claim 1.
